SEQUENCE LISTING (3 sequences)

SEQ ID NO: 1 - protein, 5 aa, Homo sapiens
APGPR

SEQ ID NO: 2 - protein, 5 aa, Rat
VPDPR

SEQ ID NO: 3 - protein, 5 aa, Rat
VPGPR

(19) United States
(12) Patent Application Publication
(10) Pub. No.: US 2011/0212887 A1
(43) Pub. Date: Sep. 1, 2011

(54) METHODS OF TREATING OBESITY USING ENTEROSTATIN

(75) Inventors: Byron Rubin, Honeoye Falls, NY (US); Peter C.M. McWilliams, San Francisco, CA (US)

(73) Assignee: Harkness Pharmaceuticals, Inc.

(21) Appl. No.: 12/859,104

(22) Filed: Aug. 18, 2010

Related U.S. Application Data
(63) Continuation of application No. 11/638,048, filed on Dec. 12, 2006, now abandoned.
(60) Provisional application No. 60/750,206, filed on Dec. 13, 2005.

(51) Int. Cl. A61K 38/08; A61P 3/04; A61P 9/12; A61P 3/10

(52) U.S. Cl. 514/4.8; 514/21.8; 514/15.7; 514/6.9

(57) ABSTRACT

The present invention provides methods of treating or preventing disorders or conditions associated with enterostatin deficiency by administering to a subject in need thereof an effective amount of enterostatin. The present invention also provides methods of selecting a subject for therapy with enterostatin. Exemplary disorders or conditions associated with enterostatin deficiency include overweight, obesity, metabolic disorders, hypertension, lipid related disorders, and type II diabetes.

This application claims the benefit of priority of U.S. provisional application No. 60/750,206, filed Dec. 13, 2005, the contents of which are hereby incorporated by reference in their entireties.

### 1. FIELD OF THE INVENTION

The present invention provides methods of treating or preventing disorders or conditions associated with enterostatin deficiency by administering to a subject in need thereof an effective amount of enterostatin. The present invention also provides methods of selecting a subject for therapy with enterostatin. The present invention further provides methods of treating a patient population, for instance, patients deficient in enterostatin by administering to patients an effective amount of enterostatin. Exemplary disorders or conditions associated with enterostatin deficiency include overweight, obesity, metabolic disorders, hypertension, lipid related disorders, and type II diabetes.

### 2. BACKGROUND OF THE INVENTION

Obesity is a complex condition that is increasingly affecting the population worldwide. According to the World Health Organization, in 1995 there were an estimated 200 million obese adults worldwide and another 18 million under-five children classified as overweight. As of 2000, the number of obese adults had increased to over 300 million. See Formiguera et al., 2004, Best Practice & Research Clinical Gastroenterology, 18:6, 1125-1146.

Overweight or obesity has been shown to increase risk for several diseases and health conditions, including hypertension, dyslipidemia (high total cholesterol or high levels of triglycerides), type II diabetes, coronary heart disease, stroke, gallbladder disease, osteoarthritis, sleep apnea and respiratory problems and some cancers (for example, endometrial, breast, and colon). See, e.g., U.S. National Center for Chronic Disease Prevention and Health Promotion. Its health consequences range from increased risk of premature death to serious chronic conditions that reduce the overall quality of life.

Various therapies have been proposed or tested for the modulation of physiological processes that might lead to conditions such as overweight or obesity. See Orzano et al., 2004, J. Am. Board Fam. Pract. 17(5):359-69. One of these is enterostatin.

Enterostatin is a peptide that has shown promise in modulating dietary fat preference in rodents. See, e.g., Erlanson-Albertsson et al., 1991, Physiol. Behav. 49:1191-1194; Okada et al., 1991, Physiol. Behav. 49:1185-1189; Shargill et al., 1991, Brain Res. 544:137-140. Enterostatin is generated by tryptic activation of procolipase in the intestine or stomach to generate colipase. Colipase binds and activates the enzyme lipase to metabolize fats in the intestine. The propeptide enterostatin is believed to reduce dietary fat preference in mammals as demonstrated in rodent studies. See, Erlanson-Albertsson et al., 1991, Okada et al., 1991, Physiol. Behav. 49:1185-1189, Shargill et al., 1991. Accordingly, studies of decreasing appetite in mammals by administering an effective amount of an enterostatin peptide have been reported. See, Erlanson-Albertsson, 1996, U.S. Pat. No. 5,494,894.

One study in human reported that the immunoreactivity of one form of enterostatin (VPDPR) appeared to be elevated in the serum of obese women and that the rise of the immunoreactivity of another form of enterostatin (APGPR) after a meal in the serum of obese women was reduced. See Prasad et al., 1999, J. Clin. Endocrinol. Metab. 84:937-941. In another study, it was reported enterostatin administered orally to humans did not affect food intake, energy expenditure or body weight. See Kovacs et al., 2003, British J. Nutrition 90:207-214.

There is a need in the art for effective methods of treating obesity and related diseases. The present invention fulfills these needs and provides such methods. The present invention is based, in part, on the discovery that by evaluating the level of enterostatin, it is possible to identify subjects that are responsive to enterostatin treatment.

### 3. SUMMARY OF THE INVENTION

In one aspect, the present invention provide methods of treating or preventing a disorder or condition associated with enterostatin deficiency in an enterostatin-deficient subject. The methods comprise the step of administering to the enterostatin-deficient subject an amount of enterostatin effective for treating or preventing the disorder or condition.

While not intending to be bound by any particular theory of operation, it is believed that the most effective approach to treat conditions such as overweight or obesity is to apply specific therapies to specific patient populations. Advantageously, in certain embodiments, the present invention provides methods of selecting a sub-population of subjects suitable for treatment with an effective amount of enterostatin according to the method described herein.

Exemplary disorders or conditions associated with enterostatin deficiency include, but are not limited to, overweight, obesity, metabolic disorders, hypertension, lipid related disorders, and type II diabetes.

In another aspect, the present invention provides methods of treating enterostatin deficiency in a subject in need thereof. The methods comprise the step of administering to the enterostatin-deficient subject an amount of enterostatin effective for treating the deficiency.

In another aspect, the present invention provides methods of selecting a subject for therapy with enterostatin. In certain embodiments, the methods comprise the step of determining the amount of enterostatin in a sample from the subject. The subject is selected for treatment when the amount of enterostatin in the sample of the subject is less than a normal enterostatin value. Normal enterostatin values are described in detail below.

In further aspect, the present invention provides for methods of treating or preventing a disorder or condition associated with enterostatin deficiency. The methods comprise the step of selecting a subject deficient in enterostatin for treatment and administering to the subject an amount of enterostatin effective for treating or preventing the disorder or condition. The methods of selecting an enterostatin-deficient subject and administering enterostatin are described herein.

In certain embodiments, a subject is enterostatin-deficient when the subject expresses or secretes a lower amount of enterostatin than a control subject does. Whether a subject is enterostatin-deficient can be determined by any method available to those of skill in the art. Exemplary methods are described herein.

The enterostatin for administration in the methods can be any peptide with enterostatin activity or F₁-ATPase activity. In some embodiments, the enterostatin is a peptide having a sequence selected from the group consisting of consisting of APGPR (SEQ ID NO:1), VPDPR (SEQ ID NO:2) and VPGPR (SEQ ID NO:3). The enterostatin can be prepared and formulated by any methods known in the art. Useful enterostatin forms and compositions are described in U.S. provisional application No. 60/750,208, filed Dec. 13, 2005, entitled “Non-Hygroscopic Compositions of Enterostatin,” and 60/750,207, filed Dec. 13, 2005, entitled “Stable Solid Forms of Enterostatin,” the contents of which are incorporated by reference in their entirety.

The enterostatin can be administered by any route known to those of skill in the art, including but not limited to orally, intravenously, intragastrically, intraduodenally, intraperitoneally or intracerebroventricularly. In certain embodiments, the enterostatin is administered in an amount of from about 1 mg/day to about 500 mg/day, from about 1 mg/day to about 400 mg/day, from about 1 mg/day to about 300 mg/day, from about 1 mg/day to about 200 mg/day, or from about 1 mg/day to about 100 mg/day.

Advantageously, the normal enterostatin value need not be determined by one carrying out a method of the invention. Instead, the normal enterostatin value can be identified by consulting knowledge or data available to those of skill in the art. Such data can be obtained from any source available to those of skill in the art. In certain embodiments, sources can be developed with the amounts of enterostatin collected by those of skill in the art according to methods described herein.

In certain embodiments, the normal enterostatin amount is from a control subject presenting no symptom of a disorder or condition associated with enterostatin-deficiency. In some embodiments, the control subject is a healthy subject with normal weight. In some embodiments, the control subject is a lean individual of normal weight.

The amount of enterostatin in the subject can be determined according to any technique known to those of skill in the art without limitation. In certain embodiments, the technique for measuring enterostatin is not critical for the invention and need not even be carried out by one practicing methods herein. In certain embodiments, the amount of enterostatin in the sample of the subject is determined by a technique described herein followed by comparing the amount to a normal enterostatin value in order to determine whether to select the subject for treatment with enterostatin. In certain embodiments, the amount of enterostatin in the sample of the subject is determined by spectrometry, chromatography, immunoassay or electrophoresis as described in detail below. In some preferred embodiments, the amount of enterostatin is determined by immunoassay. In one preferred embodiment, the immunoassay is ELISA. In some preferred embodiments, the amount of enterostatin is determined by electrophoresis. In one preferred embodiment, the immunoassay is CGE.

The amount of enterostatin can be measured in any sample of the subject as provided herein. The sample can be a fluid or tissue sample as described herein. Processes for preparing the fluid or tissue, for example, processes for extracting or purifying enterostatin are described herein.

In another aspect, the present invention provides for kits for selecting a subject for treatment of obesity with enterostatin. In some embodiments, the kits comprise a device capable of containing a fluid of the subject and a reagent capable of detecting enterostatin in the fluid. The kits can further comprise a label or labeling with instructions for using the kits. In certain embodiments, the kits comprise a label or labeling with a normal enterostatin value.

### 4. DETAILED DESCRIPTION OF THE INVENTION

### 4.1. Definition

As used herein, the following terms shall have the following meanings:

The term “subject” refers to animals such as mammals, including, but not limited to, primate (e.g., human), cow, sheep, goat, horse, dog, cat, rabbit, rat, mouse and the like. In preferred embodiments, the subject is a human.

The term “enterostatin” encompasses the propeptide of procolipase, as is known to those of skill in the art. Exemplary enterostatins have an amino acid sequence selected from the group consisting of APGPR (SEQ ID NO:1), VPDPR (SEQ ID NO:2) and VPGPR (SEQ ID NO:3). In a preferred embodiment, the enterostatin has an amino acid sequence of APGPR (SEQ ID NO:1).

The term that a subject is “enterostatin-deficient” or a subject has “enterostatin deficiency” refers to a subject that expresses or secretes a lower amount of enterostatin than expected for the subject according to the judgment of one of skill in the art. In some embodiments, a subject is “enterostatin-deficient” when the subject does not express or secrete an amount of enterostatin that is detectable using techniques available in the art. In some embodiments, a subject is “enterostatin-deficient” when the subject does not express or secrete any enterostatin.

In certain embodiments, a subject is “enterostatin-deficient” when the subject expresses or secretes a lower amount of enterostatin in a fasting state than a control subject does. In certain embodiments, a subject is “enterostatin-deficient” when expresses or secretes a lower amount of enterostatin after a meal than a control subject does. The meal can be a regular meal or a high fat meal. In some embodiments, the meal contains about 600, 700, 800, 900, 1000, 1100 or 1200 calories. In some embodiments, the meal contains about 20, 30, 40, 35, 45, 50, 55, 60 or 65% energy from fat. In one embodiment, the meal contains about 800 calories and about 45% energy from fat.

Whether a subject is “enterostatin-deficient” can be determined by techniques known to those of skill in the art. In certain embodiments, it is determined by measuring the amount of enterostatin in a sample from the subject and comparing such with a normal enterostatin value. Preferably, the sample is obtained from the subject about one, two, or three hours after a high fat meal. In some embodiments, the sample is taken from the subject and measured continuously during a period, for example, a three-hour period after a meal. In some embodiments, the subject is “enterostatin-deficient” when the amount of enterostatin in the sample from the subject is less than 95%, 90%, 85%, 80%, 75%, 70%, 65%, 60%, 55%, 50%, 45%, 40%, 35%, 30%, 25%, 20%, 15%, 10%, 5%, 2%, or 1% of a normal enterostatin value according to the judgment of a practitioner of skill in the art.

The term a “control subject” is a subject that presents no symptoms of one or more disorders or conditions associated with enterostatin-deficiency according to standards recognized by those of skill in the art. In some embodiments, a control subject has age, height, race and gender similar to a subject to be selected for treatment with enterostatin. In some embodiments, the control subject is a lean subject or a subject with normal weight. When the subject is human, the control subject can be an individual with a BMI range of 20-25 kg/m². A control subject is useful for establishing a normal enterostatin value that can be used to evaluate whether a subject is enterostatin-deficient.

“Preventing” or “prevention” refers to a reduction in the risk of acquiring a disease or disorder (i.e., causing at least one of the clinical symptoms of the disease not to develop in a subject that may be exposed to or predisposed to the disease but does not yet experience or display symptoms of the disease). Preferably, prevention refers to the use of a compound or composition in a subject not yet affected by the disease or disorder or not yet exhibiting a symptom of the disease or disorder, for instance a subject not yet infected or not yet exhibiting the symptoms of infection.

“Treating” or “treatment” of any disease or disorder refers, in one embodiment, to ameliorating the disease or disorder (i.e., arresting or reducing the development of the disease or at least one of the clinical symptoms thereof) that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

The term “an effective amount” means an amount of enterostatin or compositions comprising enterostatin that when, administered to a subject for treating a disease is sufficient to effect such treatment for the disease. An effective amount can vary depending on, inter alia, the enterostatin used, the disease and its severity, and the age, weight, etc. of the subject to be treated.

The term “obesity” refers to a subject having weight and body mass particularly of fat tissue above currently accepted standards. In some embodiments, the subject is obese with a Body Mass Index (“BMI”) above currently accepted standard. BMI is obtained by dividing body weight (in kilograms) by the height (in meters) squared. When a subject is a human, the current standards for both men and women accepted as “normal” are a BMI of 20-24.9 kg/m². In such embodiments, an obese subject has a BMI of 30 kg/m² or greater. In some embodiments, an obese subject has a BMI of 40 kg/m² or greater. In other embodiments, the subject is obese when it weighs more than 120% of the normal body weight for its age and height. Normal body weights vary among species and individuals based on height, body build, bone structure and sex.

The term “overweight” refers to a moderate excess of fat in a subject. In some embodiments, when a subject is a human, the overweight subject has a BMI of 25 kg/m² or greater. Body mass index (BMI) is obtained by dividing body weight (in kilograms) by the height (in meters) squared.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter amino acid abbreviations designate amino acids in the L-configuration. Amino acids in the D-configuration are preceded with a “D-.” For example, Arg designates L-arginine and D-Arg designates D-arginine. Likewise, the capital one-letter abbreviations refer to amino acids in the L-configuration. Lower-case one-letter abbreviations designate amino acids in the D-configuration. For example, “R” designates L-arginine and “r” designates D-arginine. Unless noted otherwise, when peptide or polypeptide sequences are presented as a series of one-letter and/or three-letter abbreviations, the sequences are presented in the N-terminal to C-terminal direction, in accordance with common practice.

In preferred embodiments, any peptide or amino acid of the invention is in the L form, unless otherwise indicated.

### 4.2. Methods of Treatment or Prevention

The present invention provides methods of treating enterostatin deficiency in a subject in need thereof. In certain embodiments, the methods comprise the step of administering to the enterostatin-deficient subject an amount of enterostatin effective for treating the deficiency. Whether a subject is enterostatin-deficient can be determined by any method available to those of skill in the art. In certain embodiments, a subject is enterostatin-deficient when the subject expresses or secretes a lower amount of enterostatin than a control subject does. Exemplary methods are described herein.

The present invention further provides methods of treating or preventing a disorder or condition associated with enterostatin deficiency in an enterostatin-deficient subject. The methods comprise the step of administering to the enterostatin-deficient subject an amount of enterostatin effective for treating or preventing the disorder or condition.

Exemplary disorders or conditions associated with enterostatin deficiency include, but are not limited to, overweight, obesity, metabolic disorders, hypertension, lipid related disorders, and type II diabetes.

In certain embodiments, the disorder or condition associated with enterostatin deficiency is overweight. In another embodiments, the disorder or condition associated with enterostatin deficiency is obesity. In certain embodiments, the disorder or condition associated with enterostatin deficiency is a metabolic disorder. In certain embodiments, the disorder or condition associated with enterostatin deficiency is a lipid related disorder. In certain embodiments, the disorder or condition associated with enterostatin deficiency is type II diabetes. In certain embodiments, the disorder or condition associated with enterostatin deficiency is hypertension.

In one aspect, the present invention provides methods of reducing appetite in an enterostatin-deficient subject in need thereof. The methods comprise the step of administering to the enterostatin-deficient subject an amount of enterostatin effective for reducing appetite.

In another aspect, the present invention provides methods of reducing food intake in an enterostatin-deficient subject in need thereof The methods comprise the step of administering to the enterostatin-deficient subject an amount of enterostatin effective for reducing food intake.

In another aspect, the present invention provides methods of reducing fat intake in an enterostatin-deficient subject in need thereof The methods comprise the step of administering to the enterostatin-deficient subject an amount of enterostatin effective for reducing fat intake.

In another aspect, the present invention provides methods of reducing body weight or stimulating weight loss in an enterostatin-deficient subject in need thereof. The methods comprise the step of administering to the enterostatin-deficient subject an amount of enterostatin effective for reducing body weight or stimulating weight loss. The term “weight loss” refers to a detectable decrease of body mass in a subject compared to the mass of the subject at a previous time.

In another aspect, the present invention provides methods of reducing body fat in a subject in need thereof The methods comprise the step of administering to the enterostatin-deficient subject an amount of enterostatin effective for reducing body fat.

In another aspect, the present invention provides methods of treating abnormal glucose levels in a subject in need thereof The methods comprise the step of administering to the enterostatin-deficient subject an amount of enterostatin effective for treating abnormal glucose levels.

It has been reported that enterostatin can induce weight loss, decrease body fat, affect appetite, decrease food intake and specifically fat intake. See also Erlanson-Albertsson et al., 1997, Obesity Research 5:4, 360-372. Therefore, the present invention also provides methods of treating or preventing various conditions or diseases associated with food intake and weight control.

### 4.2.1 Subjects

In certain embodiments of the invention, the subject is an animal, preferably a mammal, more preferably a non-human primate. In the most preferred embodiments, the subject is a human. The subject can be a male or female subject.

The methods of the invention can be used for selecting a subject for therapy with enterostatin in any subject. Particularly useful subjects include those that are enterostatin-deficient. Whether a subject is enterostatin-deficient can be determined by any methods available to those of skill in the art. Exemplary methods are described below.

In certain embodiments, the subject is at risk for a disorder or condition associated with enterostatin deficiency including, but not limited to, overweight, obesity, metabolic disorders, hypertension, lipid related disorders, and type II diabetes. In some embodiments, the subject is at risk for overweight or obesity. In some embodiments, the subject is at risk for a metabolic disorder. In some embodiments, the subject is at risk for hypertension. In some embodiments, the subject is at risk for a lipid related disorder. In some embodiments, the subject is at risk for type II diabetes.

In certain embodiments, the subject is not healthy. In some embodiments, the subject has diabetes, gastrointestinal and/or cardiovascular diseases. In some embodiments, the subject has or suffers from a disorder or condition associated with enterostatin deficiency including, but not limited to, overweight, obesity, metabolic disorders, hypertension, lipid related disorders, and type II diabetes. In some embodiments, the subject has a metabolic disorder. In some embodiments, the subject suffers from hypertension. In some embodiments, the subject has a lipid related disorder. In some embodiments, the subject has type II diabetes. In some embodiments, the subject has abnormal glucose levels.

In some embodiments, the subject is overweight. In particular embodiments, the subject is a human and has a BMI of 25 kg/m² or greater. In some embodiments, the subject is a human and has a BMI between 25 kg/m² and 30 kg/m². In some embodiments, the subject is obese. In some embodiments, the subject is a human and has a BMI of 30 kg/m² or greater. In some embodiments, the subject is a human and has a BMI between 30 kg/m² and 35 kg/m². In some embodiments, the subject is a human and has a BMI of 35 kg/m² or greater. In some embodiments, the subject is a human and has a BMI of 40 kg/m² or greater. In some embodiments, the subject weighs more than 120% of the normal weight for its age and height. In some embodiments, the subject is a human and weighs more than 96 kg.

In some embodiments, the subject is a human and has a waist circumference greater than 1.02 m. In some embodiments, the subject is a human and has a hip circumference greater than 1.06 m. In some embodiments, the subject is a human and has a waist: hip ratio greater than 0.98. In some embodiments, the subject is a human and has more than 40.9% body fat.

In some embodiments, the subject is within the normal weight range. In the context of this invention, the subject with normal weight include those subjects that, for any reason according to the judgment of a practitioner of the art, are in need of treatment with enterostatin. In some embodiments, the subject is a human and has a BMI of 25 kg/m² or less. In some embodiments, the subject is a human and has a BMI of 22 kg/m² or less. In some embodiments, the subject is a human and has a BMI between about 20 kg/m² and about 25 kg/m². In some embodiments, the subject is a human and has a BMI of 20 kg/m² or less. Such a subject could have enterostatin deficiency with his or her weight maintained, for example, by a condition, such as bulimia.

In some embodiments, the subject has not previously undergone any treatment for a disorder or condition associated with enterostatin deficiency. In other embodiments, the subject has previously undergone or is now undergoing treatment for one or more disorders or conditions associated with enterostatin deficiency. In certain embodiments, the subject has previously undergone or is now undergoing treatment with enterostatin for such. In certain embodiments, the subject has previously undergone or is now undergoing treatment other than enterostatin.

In some embodiments, the subject has abnormal glucose levels. In particular embodiments, the subject has a high blood glucose level. In some embodiments, the subject has diabetes. In certain embodiments, the subject has type II diabetes. In other embodiments, the subject does not have diabetes.

In some embodiments, the subject is below 21 years old. In some embodiments, the subject is below 15 years old. In other embodiments, the subject is more than 49 years old. In some embodiments, the subject is more than 15, 25, 35, 40, 45, 50, 55, or 65 years old.

In some embodiments, the subject exercises regularly. In other embodiments, the subject does not exercise regularly.

### 4.3. Methods of Selecting Subjects for Treatment with Enterostatin

The present invention is based, in part, on the discovery that treatments of obesity and related diseases with enterostatin can be effective in subjects that are responsive to enterostatin treatment, and subjects that are responsive to enterostatin treatment include those that are deficient in endogenous levels of enterostatin.

### 4.3.1 Determining the Level of Enterostatin

The endogenous level of enterostatin in a subject can be determined by any method available to those of skill in the art. It can be determined directly or indirectly. In some embodiments, it is determined from measuring the amount of enterostatin in a sample from a subject. In other embodiments, it is determined from measuring the activity of procolipase, the precursor of enterostatin, in a sample from a subject.

In certain embodiments of the invention, the method of determining the amount of enterostatin is not critical. Accordingly, the present invention provides methods for selecting a subject for treatment with enterostatin that comprise the single step of determining whether a subject is suitable for treatment based on the amount of enterostatin in a sample from the subject.

The amount of the enterostatin can be determined by one practicing a method of the invention in any manner whatsoever. Exemplary techniques are described herein.

The amount of enterostatin can be determined from any sample from the subject, which can be, by way of example and not of limitation, a blood sample, a plasma sample, a saliva sample, a serum sample, a sputum sample, a urine sample, a stool sample, a cell sample, a cellular extract sample, a tissue biopsy sample or any sample that may be obtained from a subject using techniques well known to those of skill in the art. The precise sample that is taken from the subject may vary, but the sampling preferably is minimally invasive and is easily performed by conventional techniques.

The sample can be processed or purified according to the judgment of those of skill in the art based on, for example, the type of sample used and the measurement technique. Particularly useful processing steps are precipitation, centrifugation, filtration and/or chromatography.

In certain embodiments, to extract and purify the enterostatin, the blood sample can be processed, for example, as described in Prasad et al., 1999, J. Clin. Endocrinol. Metab. 84:937-941, the contents of which are incorporated by reference in its entirety. For instance, 5 ml blood can be collected from a subject. The blood sample can be centrifuged and processed for the analysis of enterostatin. The blood sample can be stored frozen at −70° C. until assayed for enterostatin. To extract enterostatin, the blood sample can be mixed with 1:9 volume methanol. The mixture can be stored over ice for 30-60 minutes and then centrifuged at 11,000 g for 10 minutes at 4° C. The clear supernatant can be lyophilized to dryness and then reconstituted appropriately for ELISA or chromatography.

In certain embodiments, to extract and purify the enterostatin, the urine sample can be processed, for example, as described in Bowyer et al. 1993, Gut, 34: 1520-1525, the contents of which are incorporated by reference in its entirety. For instance, 10 ml urine sample from the subject can be collected. The urine sample can be mixed with 20 mM zinc acetate. To minimize loss of enterostatin immunoreactivity on storage, each urine sample can be centrifuged at 3000 g for 20 minutes, the supernatant suspended in a boiling bath for 10 minutes, centrifuged for five minutes at 10,000 g and the supernatant aliquoted and stored at −20° until assayed.

To analyze the enterostatin extracted, the stored aliquots of urine or blood sample can be thawed at room temperature, thoroughly whirl-mixed, centrifuged for five minutes at 10,000 g, and the supernatant assayed. Gel filtration chromatography can be performed using Sephadex G-25 (50×1.0 cm; 39.3 ml; fractionation range for globular proteins, 1-5 kDa) column. Lyophilized methanol-extracted sample reconstituted in a minimal volume of distilled water can be loaded on columns equilibrated with buffer A (10 mmol/l NH4HCO3).

### 4.3.1.1. Measuring the Amount of Enterostatin

The amount of enterostatin in a sample from a subject can be determined by any methods known to those of skill in the art without limitation. For example, it can be determined by spectrometry, chromatography, immunoassay or electrophoresis In some embodiments, the amount of enterostatin is determined by immunoassay. In some preferred embodiments, the amount of enterostatin is determined by ELISA such as described by Imamura et al, 1998, Peptides, 19:8, 1385-1391; Bowyer et al., 1991, Clinica. Chimica. Acta. 200:137-152 (for APGPR); Mizuma et al., 1995, Biochemical & Biophysical Research Communications 1995, 215(1): 227-234 (for VPDPR); the contents of which are incorporated by reference in their entirety. In other preferred embodiments, the amount of enterostatin is determined by capillary gel electrophoresis (“CGE”) as described in Zhao et al., 2001, Fresenius J. Anal. Chem., 269:220-224, the contents of which are incorporated by reference in its entirety.

Standard techniques for determining the amount of a peptide or a peptide of interest present in a sample may be utilized for determining the amount of enterostatin in a sample. For example, standard techniques can be employed using, e.g., immunoassays such as, for example Western blot, immunoprecipitation followed by sodium dodecyl sulfate polyacrylamide gel electrophoresis, (SDS-PAGE), immunocytochemistry, and the like to determine the amount of protein or proteins of interest present in a sample. One exemplary agent for detecting a protein of interest is an antibody capable of specifically binding to a protein of interest, preferably an antibody detectably labeled, either directly or indirectly.

For such detection methods, if desired the enterostatin from the sample can easily be isolated using techniques which are well known to those of skill in the art. Those methods can, for example, be such as those described in Harlow and Lane, 1988, Antibodies: A Laboratory Manual. Cold Spring Harbor Laboratory Press (Cold Spring Harbor, N.Y.), which is incorporated by reference herein in its entirety.

In certain embodiments, methods of the amount of enterostatin in a sample involve detection via interaction with an enterostatin-specific antibody. Antibodies can be generated utilizing standard techniques well known to those of skill in the art. In specific embodiments, antibodies can be polyclonal, or more preferably, monoclonal. An intact antibody, or an antibody fragment (e.g., scFv, Fab or F(ab′)₂) can, for example, be used. Exemplary immunoassays are described below.

In some embodiments, a protein chip assay (See, e.g., Zhu & Snyder, 2003, Curr. Opin. Chem. Biol. 7:55-63; Mitchell, 2002, Nature Biotechnology 20:225-229) is used to measure amounts for the biomarkers in the biomarker profile. See also, for example, Lin, 2004, Modern Pathology, 1-9; Li, 2004, Journal of Urology 171, 1782-1787; Wadsworth, 2004, Clinical Cancer Research, 10, 1625-1632; Prieto, 2003, Journal of Liquid Chromatography & Related Technologies 26, 2315-2328; Coombes, 2003, Clinical Chemistry 49, 1615-1623; Mian, 2003, Proteomics 3, 1725-1737; Lehre et al., 2003, BJU International 92, 223-225; and Diamond, 2003, Journal of the American Society for Mass Spectrometry 14, 760-765, which are hereby incorporated by reference in their entireties. Particularly useful in certain embodiments of the invention are antibody chips that facilitate detection by MALDI or SELDI (See, e.g., Wang, et al., 2001, Int'l. J. of Cancer 92:871-876; Figeys, 2002, Proteomics 2:373-382; Sonksen et al., 1998, Anal. Chem. 70:2731-6; Glökler, & Angenendt, 2003, J. Chromatography B, 797:229-240; the contents of which are hereby incorporated by reference in their entireties).

In certain embodiments, antibodies, or fragments of antibodies, specific for enterostatin can be used to determine the amount of enterostatin in a sample. This can be accomplished, for example, by immunofluorescence techniques. Antibodies (or fragments thereof) can, additionally, be employed histologically, as in immunofluorescence or immunoelectron microscopy, for in situ determination of enterostatin.

Immunoassays typically comprise incubating a sample of a detectably labeled antibody capable of identifying enterostatin, and detecting the bound antibody by any of a number of techniques well-known in the art. Exemplary immunoassays are Western plot, immunoprecipitation followed by sodium dodecyl sulfate polyacrylamide gel electrophoresis (SDS-PAGE), immunocytochemistry and the like to the determine the amount of a peptide in a sample.

One of the ways in which an antibody specific for enterostatin can be detectably labeled is by linking the same to an enzyme and use in an enzyme immunoassay (EIA) (Voller, 1978, “The Enzyme Linked Immunosorbent Assay (ELISA)”, Diagnostic Horizons 2:1-7, Microbiological Associates Quarterly Publication, Walkersville, Md.; Voller et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo, each of which is hereby incorporated by reference in its entirety). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection can also be accomplished by visual comparison of the extent of enzymatic reaction of a substrate in comparison with similarly prepared standards.

Measurement can also be accomplished using any of a variety of other immunoassays. For example, by radioactively labeling the antibodies or antibody fragments, it is possible to detect a biomarker through the use of a radioimmunoassay (RIA) (See, for example, Weintraub, B., Principles of Radioimmunoassays, Seventh Training Course on Radioligand Assay Techniques, The Endocrine Society, March, 1986, which is incorporated by reference herein). The radioactive isotope (e.g., ¹²⁵I, ¹³¹I, ³⁵S or ³H) can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The amount of enterostatin can also, for example, be determined by the use of one or more of the following methods described below. For example, methods may include nuclear magnetic resonance (NMR) spectroscopy, a mass spectrometry method, such as electrospray ionization mass spectrometry (ESI-MS), ESI-MS/MS, ESI-MS/(MS)ⁿ (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, atmospheric pressure photoionization mass spectrometry (APPI-MS), APPI-MS/MS, and APPI-(MS)ⁿ. Other mass spectrometry methods may include, inter alia, quadrupole, Fourier transform mass spectrometry (FTMS) and ion trap. Other suitable methods may include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE) or other chromatography, such as thin-layer, gas or liquid chromatography, or any combination thereof In one embodiment, the biological sample may be fractionated prior to application of the separation method.

In one embodiment, laser desorption/ionization time-of-flight mass spectrometry is used to determine the amount of a biomarker where the biomarker is a molecule that has been ionized and vaporized off an immobilizing support by incident laser radiation. A variety of laser desorption/ionization techniques are known in the art (See, e.g., Guttman et al., 2001, Anal. Chem. 73:1252-62 and Wei et al., 1999, Nature 399:243-246, which are hereby incorporated by reference).

Laser desorption/ionization time-of-flight mass spectrometry allows the generation of large amounts of information in a relatively short period of time. A biological sample is applied to one of several varieties of a support that binds all of the biomarkers, or a subset thereof, in the sample. Cell lysates or samples are directly applied to these surfaces in volumes as small as 0.5 μL, with or without prior purification or fractionation. The lysates or sample can be concentrated or diluted prior to application onto the support surface. Laser desorption/ionization is then used to generate mass spectra of the sample, or samples, in as little as three hours.

Analysis by liquid chromatography-mass spectrometry produces a mass intensity spectrum, the peaks of which represent various components of the sample, each component having a characteristic mass-to-charge ratio (m/z) and retention time (r.t.). The presence of a peak with the m/z and retention time of a biomarker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. In addition, deconvoluting software is available to separate overlapping peaks. The retention time depends to some degree on the conditions employed in performing the liquid chromatography separation.

In MALDI mass spectrometry (MALDI-MS), various mass analyzers can be used, e.g., magnetic sector/magnetic deflection instruments in single or triple quadrupole mode (MS/MS), Fourier transform and time of flight (TOF), including orthogonal time-of-flight (O-TOF), configurations as is known in the art of mass spectrometry. For the desorption/ionization process, numerous matrix/laser combinations can be used. Iontrap and reflectron configurations also can be employed.

Electrospray ionization mass spectrometry (ESI-MS) is broadly applicable for analysis of macromolecules, including proteins, nucleic acids, and carbohydrates (Fenn et al., 1989, Science 246:64-71; Crain et al., 1998, Curr. Opin. Biotechnol. 9:25-34; Smith et al., 1990, Anal Chem. 62:882-99; Han & Gross, 1994, Proc Natl Acad Sci USA 91: 10635-10639). Electrospray techniques have been used to separate and measure biomarkers like those of formula I and formula Ia (See Petkovic et al., 2001, Anal Biochem. 289(2):202-16; Pulfer & Murphy, 2003, Mass Spec Rev 22:332-364; Han & Gross, 1995, J. Amer. Soc. Mass Spec. 6:1202-1210; the contents of which are hereby incorporated by reference in their entireties).

For proteins or peptides, Vorm, O. et al., Anal. Chem. 66:3281-3287 (1994); and Vorm and Mann, J. Am. Soc. Mass. Spectrom. 5:955-958 (1994)), for example, provide additional guidance on mass spectral analysis of such molecules and are incorporated by reference in their entirety. The contents of these publications are hereby incorporated by reference in their entireties.

### 4.3.1.2. Measuring the Activity of Procolipase

In addition to direct measurement of endogenous level of enterostatin, those of skill in the art will understand that the level of enterostatin can also be determined from the activity of procolipase or its cleavage product, colipase.

Procolipase, the parent molecule of enterostatin, is inactive before secreted into pancreatic juice. In the presence of trypsin in pancreatic juice, procolipase is cleaved to form the active colipase and an amino-terminal pentapeptide, i.e., enterostatin. The role of colipase is to bind to lipase, thus activating pancreatic lipase. See Erlanson-Albertsson et al., 1997, Obesity Research 5:4, 360-372. The activity of procolipase can be used as an index of the level of enterostatin in a subject.

Procolipase activity can be determined by any methods known to those of skill in the art. For example, it can be measured in the duodenal content as described in Erlanson-Albersson et al., 1987, Regul. Pept. 22:325-331; Okada et al., 1992, Am. J. Physiol. 262(6 pt 2):R1111-16, the contents of which are incorporated by reference in its entirety.

### 4.3.2 Selecting Subjects for the Treatment with Enterostatin

In certain embodiments, subjects with low levels of enterostatin or subjects with enterostatin deficiency are selected for treatment. The subject is selected for treatment when the amount of enterostatin in the sample of the subject is less than a normal enterostatin value. In some embodiments, a subject that does not express or secrete an amount of enterostatin detectable using techniques available in the art is selected. In other embodiments, the subject that does not express or secrete any enterostatin is selected. In preferred embodiments, a subject is selected when the subject expresses or secretes a lower amount of enterostatin after a high fat meal than a control subject does. The low level of the enterostatin in a subject can be due to any cause known in the art. For example, it may be due to low level of expression or secretion of enterostatin, or due to inadequate tryptic activation of procolipase in the intestine or stomach. It can also be a consequence of excessive proteolytic activity, such as excessive protease activity that can hydrolyze enterostatin, e.g., peptidase dipeptidyle peptidase IV (DPPIV).

In certain embodiments, the selection can be based on the amount of enterostatin in a sample of the subject and a normal enterostatin value. Normal enterostatin values are described in the section below. In some embodiments, if the amount of enterostatin in the test subject is below, or substantially below, the normal enterostatin value, the test subject is selected for treatment with enterostatin. In some embodiments, the subject is selected when the amount of enterostatin in the sample from the subject is less than a normal enterostatin value. In other embodiments, a subject is selected when the amount of enterostatin in the sample from the subject is less than 95%, 90%, 85%, 80%, 75%, 70%, 65%, 60%, 55%, 50%, 45%, 40%, 35%, 30%, 25%, 20%, 15%, 10%, 5%, 2%, or 1% of a normal enterostatin value.

In certain embodiments, one or more samples taken at a single point in time from the subject are used to make the selection. In some embodiments, only a single sample at a single time point is taken. In other embodiments, a plurality of samples taken at a single time point from the subject are taken. A plurality of samples can be same or different sample types. In particular embodiment, both a blood sample and a urine sample are taken from the subject to make the selection. When a plurality of the same type of samples are used, the evaluation can be based on any statistical technique know to those of skill in the art, such as ANOVA or Chi squared test.

When a sample at a single time point is used, the sample can be obtained from the subject when the subject is fasted overnight, when the subject is fed, or about 0.5, 1.0, 1.5, 2.0, 2.5, 3.0, 3.5 or 4.0 hours after the subject is fed. In some embodiments, the subject is fed with a high-fat meal or a regular meal. In some embodiments, the high fat meal contains about 600, 700, 800, 900, 1000, 1100, or 1200 calorie. In some embodiments, the high fat meal contains about 35, 45, 50, 55, 60, or 65% energy as fat. In one embodiment, the high-fat meal contains about 800 cal and about 45% energy as fat.

In certain embodiments, a plurality of samples taken at different points in time from the subject are used to make the selection. The times can be separated according to the judgment of those of skill in the art of skill in the art. In some embodiments, these samples are obtained from the subject either on a daily basis, or alternatively more frequently, e.g., every 4, 6, 8, or 12 hours.

In some embodiments, a plurality of samples taken at different time points is for purpose of repeated measurement. In such embodiments, the evaluation can be based on any statistical technique know to those of skill in the art, such as ANOVA or Chi squared test. Preferably, the samples are taken from the subject when the subject is under the same or similar feeding conditions according to the judgment of a practitioner of skill. In some embodiments, all of samples are taken when the subject is fasted overnight. In some embodiments, all of samples are taken when the subject is fed. In some embodiments, all of samples are taken about a particular time after the subject is fed. In particular embodiments, all of samples are taken about 0.5, 1.0, 1.5, 2.0, 2.5, 3.0, 3.5 or 4.0 hours after the subject is fed with a high fat meal.

In other embodiments, a plurality of samples taken at different time points from the subject and a change or no change in the amount of enterostatin is evaluated to make the selection. It has been reported that obese subjects not only have reduced endogenous level of enterostatin but also have a impaired meal-induced increase in enterostatin. See Prasad et al., 1999, J. Clin. Endocrinol. Metab. 84(3):947-941. The diminution in the meal-induced secretion of enterostatin in obesity suggests a delay in the appearance of satiety, leading to increased caloric intake. Accordingly, the ratio of fed:fast of the amount of enterostatin can be determined and used to select a subject for treatment with enterostatin.

In certain embodiments, samples are taken both when the subject is fasted overnight, and when the subject is fed or over the course of one, two, or three hours after the subject is fed with a regular meal or a high-fat meal, and the ratio of fed:fast of the amount of enterostatin from the subject is calculated. In preferred embodiments, samples are taken over the course of three hours after the subject is fed with a high fat meal. In certain embodiments, the enterostatin levels are measured continuously for one, two or three hours following the meal. In some embodiments, a subject is selected when the ratio of fast: fed is reduced relative to a normal enterostatin fast: fed ratio. In another embodiment, a subject is selected when the ratio of fast: fed is less than 95%, 90%, 85%, 80%, 75%, 70%, 65%, 60%, 55%, 50%, 45%, 40%, 35%, 30%, 25%, 20%, 15%, 10%, 5%, 2%, or 1% of a normal enterostatin fast: fed ratio. The normal fed:fast ratio of enterostatin is described below.

In addition to the level of enterostatin, other parameters or variables can be used in combination with the level of enterostatin to select a subject for treatment with enterostatin. In some embodiments, the blood glucose level of the subject is used. In other embodiments, the body weight or BMI of the subject is used to make the selection. Further, whether a subject is selected for treatment with enterostatin may be in accordance with the judgment of those of skill in the art, for instance, based on the blood testing, eletrocardiograms, fasting chemistry panel, CBC, blood pressures, pulse rates, urinalysis and adverse events of the subject in combination with the level of enterostatin.

### 4.3.3 Normal Enterostatin Value and Normal Enterostatin Fed:Fast Ratio

The normal enterostatin value can be, for example, the amount of enterostatin in a sample from a control subject or a plurality of control subjects. The amount of enterostatin in a control subject or control subjects can be measured according to techniques known to those of skill in the art including those described herein. Advantageously, in certain embodiments, the amount of enterostatin in control subject and the amount of enterostatin in the test subject are obtained by the same technique. Those of skill in the art would understand that a normal enterostatin value may vary for each particular assay, each sample type, and each type of cell-free extract. Those of skill in the art would understand that a normal enterostatin value may vary depending on different species or gender of subjects to be selected. For example, a normal enterostatin value may be higher for a male subject than a female subject of the same species. Accordingly, in preferred embodiments, the enterostatin level for a female subject is compared to the level expected for a female subject; the enterostatin level for a male subject is compared to the level expected for a male subject.

The control subject can be a lean subject or a subject with normal weight. When the subject is human, the control subject can be an individual or individuals with normal BMI range of 20-25 kg/m². In certain embodiments, the normal enterostatin amount is from a plurality of control subjects presenting no symptom of the disorder or condition associated with enterostatin-deficiency. The normal enterostatin amount can be calculated according to any suitable statistical method known to those of skill in the art. For instance, the normal enterostatin amount can be based on the statistical mean of the enterostatin amount in samples from control subjects presenting no symptom of the disorder or condition associated with enterostatin-deficiency.

Advantageously, the normal enterostatin value need not be obtained or measured by a practitioner of a method of the invention. Instead, the amount of the normal enterostatin value can be identified by consultation in sources available to those of skill in the art, such as scientific literature, public or private databases, or by reference to the data provided herein.

The normal enterostatin value can be an absolute value, an absolute value with a margin of error or a range of values, as determined by those of skill in the art. In certain embodiments, the selection is made based on a range of normal values for the amount of enterostatin. The range of normal values can be obtained as described herein and made available to a practitioner of the methods of the invention.

In certain embodiments, the normal enterostatin value is a cutoff reference amount. A cutoff reference amount is an absolute value for the normal enterostatin amount. For instance, a cutoff reference amount of 30 nM for enterostatin in blood after a high fat meal can indicate a normal enterostatin value. The test subject is enterostatin-deficient when the test subject has an amount of the enterostatin less than 30 nM in its blood sample taken during the three hours after a high fat meal. Cutoff reference amounts can be determined using statistical techniques known to those of skill in the art based on control amounts obtained from control subjects. For instance, it can be based on the statistical mean of the amount of enterostatin in samples from control subjects.

The amount of enterostatin in a sample from a subject can be compared with a normal enterostatin value according to any suitable statistical method known to those of skill in the art. In preferred embodiments, two- or three-way analysis of variance (ANOVA) or Chi squared test is used for comparison with repeated measurement.

In certain embodiments, the subject is selected for therapy with enterostatin, based on the fed:fast ratio of enterostatin in a sample of the subject and a normal enterostatin fed:fast ratio. The fed:fast ratio of enterostatin is obtained by dividing the amount of enterostatin in a sample from a subject when the subject is fast overnight by the amount of enterostatin in the sample when the subject is fed or about 0.5, 1, 1.5, 2, 2.5, or 3 hour after the subject is fed. The above description regarding normal enterostatin values also applies to normal enterostatin fed:fast ratios. For instance, the normal enterostatin fed:fast ratio can be, for example, the normal enterostatin fed:fast ratio from a control subject or a plurality of control subjects, and it need not be obtained or measured by a practitioner of a method of the invention. It may vary for each particular assay, each sample type, and each type of cell-free extract. It can be an absolute value, an absolute value with a margin of error, a range of values, or a cutoff reference amount.

### 4.4. The Enterostatin for Administration

The enterostatin can be any enterostatin known to those of skill in the art. The enterostatin can be from the same species as a subject to be treated, or the enterostatin can be from a different species. In preferred embodiments, the enterostatin is from the same species as the subject. Exemplary enterostatins include human, rat, mouse, porcine, canine and equine enterostatin. In some embodiments, the subject is a human and the enterostatin having amino acid sequence APGPR (SEQ ID NO:1) is administered.

In certain embodiments, the enterostatin is a full-length enterostatin. Exemplary enterostatins have an amino acid sequence selected from the group consisting of APGPR (SEQ ID NO:1), VPDPR (SEQ ID NO:2) and VPGPR (SEQ ID NO:3). The enterostatin administered in the methods of the invention can comprise a single enterostatin, or can comprise multiple enterostatins. Preferred is APGPR (SEQ ID NO:1). Methods of making the enterostatins are described in detail below.

In preferred embodiments, the enterostatin is substantially pure. In this context the term “substantially pure” indicates that the enterostatin is substantially free of contaminants not intended to be administered. Examples include peptide or amino acid contaminants and peptide synthesis reagents. In certain embodiments, the enterostatin is 85%, 90%, 91%, 92%, 93%, 94%, 95%, 96%, 97%, 98%, 99%, 99.5% or 99.9% pure. As discussed in detail the section below, enterostatin can be formulated for administration with one or more carriers, excipients or diluents.

The enterostatin can comprise free termini or blocked termini according to the judgment of those of skill in the art. Useful blocked termini include a C-terminal amide or an N-terminal acetyl, or both. In preferred embodiments, the enterostatin has free N- and C-termini.

The enterostatin used in the invention can comprise a single enterostatin, or can comprise multiple enterostatins. For example, the enterostatin can be a combination of full length enterostatins with different amino acid sequence.

The enterostatin can be in a neutral form, or in a salt form. The salt form can be any salt form known to those of skill in the art. Particularly useful salt forms are those that are coordinated with acetate, chloride, sulfate and phosphate. Acetate and chloride salts are preferred.

The enterostatin can be any form of enterostatin known to those of skill in the art. The enterostatin can be in the form of a co-complex. In some embodiments, the enterostatin is in the form of a co-complex, as described in U.S. provisional application No. 60/750,208, filed Dec. 13, 2005, the contents of which are incorporated by reference in its entirety.

The enterostatin for administration in the methods of the present invention can be in any enterostatin formulation or composition apparent to those of skill in the art. Exemplary pharmaceutical formulations and compositions are described in details below. Particularly advantageous compositions are those described in U.S. provisional application No. 60/750,207, filed Dec. 13, 2005, the contents of which are incorporated by reference in its entirety.

The enterostatin can be prepared, formulated and administered to a subject by any methods apparent to those of skill in the art as described below.

### 4.5. Preparation of Enterostatin for Treatment

Enterostatin can be prepared according to any technique apparent to those of skill. Exemplary techniques for the preparation of enterostatin are described in U.S. Pat. No. 5,494,894, the contents of which are hereby incorporated by reference in its entirety. In certain embodiments, enterostatin can be prepared synthetically, for example by solution phase or solid phase peptide synthesis. See Merrifield, 1963, J. Am. Chem. Soc. 85:2149; Fields et al., 1990, Int J Pept Protein Res. 35:161-214; Fields et al., 1991, Pept Res. 4:95-101; the contents of which are hereby incorporated by reference in their entirety.

In further embodiments, enterostatin can be obtained from natural sources, recombinant sources or commercial sources. In some embodiments, the enterostatin can be obtained by recombinantly expressing procolipase, cleaving procolipase to produce enterostatin and then purifying the enterostatin.

The enterostatin used in the present invention can be purified by any art-known technique such as high performance liquid chromatography, ion exchange chromatography, gel electrophoresis, affinity chromatography and the like. The actual conditions used to purify a particular enterostatin will be apparent to those having skill in the art.

### 4.6. Formulation and Route of Administration of Enterostatin

The enterostatin for use in the treatment may be administered to a subject per se, in the form of a pharmaceutical composition, in a form of a co-complex, or in a form of a pharmaceutical composition comprising a co-complex.

The enterostatin can be administered by any route according to the judgment of those of skill in the art, including but not limited to orally, intravenously, intragastrically, intraduodenally, intraperitoneally or intracerebroventricularly.

In some embodiments, a composition comprising one or more enterostatin co-complex, or a pharmaceutically acceptable salt, solvate or hydrate thereof is administered. Useful enterostatin co-complexes and/or compositions are described in U.S. provisional application Nos. 60/750,208, filed Dec. 13, 2005, and 60/750,207, filed Dec. 13, 2005, the contents of which are incorporated by reference in their entirety. In preferred embodiments, a pharmaceutical composition or a single unit dosage form comprising enterostatin or enterostatin co-complex is administered. In a specific embodiment, a composition comprising one or more co-complexes, or a pharmaceutically acceptable salt, solvate, or hydrate thereof is administered.

In a preferred embodiment, a composition for administration is a pharmaceutical composition or a single unit dosage form. Pharmaceutical compositions and single unit dosage forms can comprise a prophylactically or therapeutically effective amount of one or more prophylactic or therapeutic agents (e.g., a co-complex comprising enterostatin, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre-gelatinized starch, and magnesium stearate.

This invention further encompasses administration of anhydrous pharmaceutical compositions and dosage forms comprising enterostatin. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses administration of pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions or single unit dosage forms are sterile and in suitable form for administration to a subject, preferably an animal subject, more preferably a mammalian subject, and most preferably a human subject.

A pharmaceutical composition comprising enterostatin is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of enterostatin will typically vary depending on their use. For example, a dosage form used in the acute treatment of a disorder may contain larger amounts of one or more of enterostatin it comprises than a dosage form used in the chronic treatment of the same disease. Also, the therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions comprising the enterostatin are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms for administration in methods of the invention comprise enterostatin or a co-complex of comprising enterostatin, or a pharmaceutically acceptable salt, solvate or hydrate thereof lie within the range of from about 0.1 mg to about 1000 mg per day, given as a single once-a-day dose in the morning but preferably as divided doses throughout the day taken with food. Particular dosage forms of the invention have about 0.1, 0.2, 0.3, 0.4, 0.5, 1.0, 2.0, 2.5, 5.0, 10.0, 15.0, 20.0, 25.0, 40.0, 50.0, 60.0, 100, 200, 250, 500 or 1000 mg of the active enterostatin.

### 4.6.1 Oral Dosage Forms

Pharmaceutical compositions used in the methods of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In preferred embodiments, the oral dosage forms are solid and prepared under anhydrous conditions with anhydrous ingredients, as described in detail in the sections above. However, the scope of the invention extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### 4.6.2 Delayed Release Dosage Forms

Enterostatin used in the methods of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461,6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 4.6.3 Transdermal, Topical & Mucosal Dosage Forms

Although solid, anhydrous oral dosage forms are preferred, the present invention also provides administration of enterostatin in transdermal, topical, or mucosal dosage forms. Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 4.6.4 Dosage & Frequency of Administration

The amount of enterostatin in the methods of the invention which will be effective in the prevention, treatment, management, or amelioration of a disorder or one or more symptoms thereof will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems.

Exemplary doses of enterostatin include milligram or microgram amounts of the active peptide per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). For enterostatin used in the invention, the dosage administered to a patient is typically 0.01 mg/kg to 15 mg/kg of the patient's body weight, based on weight of the active peptide. Preferably, the dosage administered to a patient is between 0.01 mg/kg and 15 mg/kg, 0.01 mg/kg and 10 mg/kg, 0.01 mg/kg and 5 mg/kg, 0.01 and 4 mg/kg, 0.01 and 3 mg/kg, 0.01 mg/kg and 2 mg/kg, 0.01 mg/kg and 1 mg/kg, 0.02 mg/kg and 1 mg/kg, 0.10 mg/kg and 2.5 mg/kg, of the patient's body weight.

In general, the recommended daily dose range of enterostatin in the methods of the invention for the conditions described herein lie within the range of from about 0.01 mg to about 1000 mg per day, as a single dose or multiple doses per day. Specifically, a total daily dose range should be from about 1 mg to about 500 mg per day, more specifically, between about 10 mg and about 200 mg per day. In managing the patient, the therapy can be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response. In certain embodiments, the enterostatin is administered in an amount of about 1 mg/day to about 500 mg/day. In some embodiments, it is administered in an amount of about 1 mg/day to about 400 mg/day. In some embodiments, it is administered in an amount of about 1 mg/day to about 300 mg/day. In some embodiments, it is administered in an amount of about 1 mg/day to about 200 mg/day. In some embodiments, it is administered in an amount of about 1 mg/day to about 100 mg/day.

The enterostatin can be administered as a single once-a-day dose or preferably as divided doses throughout a day. In some embodiments, the daily dose is administered twice daily in equally divided doses. In other embodiments, the daily dose is administered three times per day. In particular embodiments, the daily dose is administered three times per day in equally divided doses. In some embodiments, the daily dose is administered three times per day in three divided doses and each dose comprises the enterostatin in an amount between about 1-100 mg, about 4-60 mg, about 4-40 mg, about 4-30 mg, about 4-25 mg, or about 4-20 mg. Preferably, the three divided doses of the enterostatin are given around three meal times each day.

The enterostatin can be administered at various times. In some embodiments, it is administered to an enterostatin-deficient subject when the subject is fasted. In some embodiments, it is administered prior to a meal. In some embodiments, it is administered during a meal. In some embodiments, it is administered after a meal.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with administration of enterostatin of the invention are also encompassed by the above described dosage amounts and dose frequency schedules. Further, when a patient is administered multiple dosages of enterostatin of the invention, not all of the dosages need be the same. For example, the dosage administered to the patient may be increased to improve the prophylactic or therapeutic effect of the co-complex or it may be decreased to reduce one or more side effects that a particular patient is experiencing.

In a specific embodiment, the dosage of enterostatin, based on weight of the active peptide, administered to prevent, treat, manage, or ameliorate a disorder, or one or more symptoms thereof in a patient is 0.01 mg/kg, 0.05 mg/kg, 0.10 mg/kg, 0.15 mg/kg, 0.20 mg/kg, 0.25 mg/kg, 0.5 mg/kg, 0.75 mg/kg, 1 mg/kg, 1.5 mg/kg, 2 mg/kg, 3 mg/kg, 4 mg/kg, 5 mg/kg, 10 mg/kg, or 15 mg/kg or more of a patient's body weight. In another embodiment, the dosage of enterostatin administered to prevent, treat, manage, or ameliorate a disorder, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In certain embodiments, administration of enterostatin in the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

In certain embodiments, the methods and compositions can be practiced as a single, one time dose or chronically. By chronic it is meant that the methods and compositions of the invention are practiced more than once to a given individual. For example, chronic administration can be multiple doses of a pharmaceutical composition administered to a subject, on a daily basis, twice daily basis, or more or less frequently, as will be apparent to those of skill in the art. Chronic administration can continue for days, weeks, months or years if appropriate according to the judgment of the practitioner of skill.

In another embodiment, the methods and compositions are practiced acutely. By acute it is meant that the methods and compositions of the invention are practiced in a time period close to or contemporaneous with the onset of an event. For example, acute administration can be a single dose or multiple doses of a pharmaceutical composition administered around the onset of a meal. In some embodiments, the meal is a high calorie or high fat meal. Acute administration can also be a single dose or multiple doses of a pharmaceutical composition administered around the onset of a craving for food, specifically a craving for fatty food. A time period close to or contemporaneous with the onset of an event will vary according to the event but can be, for example, within about 30 minutes of a meal or a craving for food. In certain embodiments, acute administration is administration within about an hour of a meal or a craving for food. In certain embodiments, acute administration is administration within about 2 hours, about 6 hours, about 10 hours, about 12 hours, about 15 hours or about 24 hours after a meal or a craving for food.

In a specific embodiment, the invention provides a method of preventing, treating, managing, or ameliorating a disorder, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more co-complexes or compositions comprising enterostatin once every 3 days, preferably, once every 4 days, once every 5 days, once every 6 days, once every 7 days, once every 8 days, once every 10 days, once every two weeks, once every three weeks, or once a month.

An effective amount of enterostatin described herein will provide therapeutic benefit without causing substantial toxicity.

Toxicity of enterostatin can be determined by standard pharmaceutical procedures in cell cultures or experimental animals, for example, by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. T he dosage of the compounds described herein lies preferably within a range of circulating concentrations that include the effective dose with little or no toxicity. The dosage may vary within this range depending upon the dosage form employed and the route of administration utilized. T he exact formulation, route of administration and dosage can be chosen by the individual physician in view of the patient's condition. (See, e.g., Fingl et al., 1996, In: The Pharmacological Basis of Therapeutics, 9.sup.th ed., Chapter 2, p. 29, Elliot M. Ross).

### 4.7. Kits for Selecting Enterostatin-Deficient Subjects

The invention also provides kits that is useful for selecting a subject for treatment with enterostatin according to the present invention. In some embodiments, the kits of the present invention comprise a reagent that is capable of detecting enterostatin in a sample from a subject. The reagent may be an antibody or functional fragment or derivative thereof (e.g., Fab, F(ab)₂, Fv or sc Fv fragments) that specifically bind enterostatin. In some embodiments, the antibodies may be detectably labeled. The reagent may be a part of an array, or the reagent may be packaged separately and/or individually. The kit may also comprise at lease one internal standard to be used in determining the amount of enterostatin.

The kits of the present invention may also include reagents such as buffers that can be used in obtaining a sample from a subject. Prevention of the action of microorganism can be ensured by the inclusion of various antibacterial and antifungal agents, for example, paraben, chlorobutanol, phenol sorbic acid, and the like. It may also be desirable to include isotonic agents such as sugars, sodium chloride, and the like.

In certain embodiments, the kits further comprise a label or labeling with instruction for carrying out a method of the invention. For example, the label of labeling can provide a normal enterostatin value. Further, the label or labeling can provide citations or links to sources of such reference amounts. In some embodiments, at lease one positive control and at least negative control are included in the kit.

The following examples are offered to illustrate this invention and are not to be construed in any way as limiting the scope of this invention.

### 5. EXAMPLES

### 5.1. Example 1

### Measuring the Amount of Enterostatin in a Blood Sample

This example provides determining the amount of enterostatin in a blood sample using ELISA. ELISA using anti-APGPR antibodies can be performed as described in Imamura et al, 1998, Peptides, 19:8, 1385-1391; Bowyer et al., 1991, Clinica. Chimica. Acta. 200:137-152, the contents of which are incorporated hereby by reference in their entirety.

Collection of Blood Samples

5 ml blood sample is collected and immediately mixed with 20 mM zinc acetate and allowed to clot at room temperature for 30 minutes. The sample is then centrifuged at 3000 g for 20 minutes. The separated serum is mixed with an equal volume of ELISA immunoassay buffer containing 50 mM TRIS/HCL, 0.05% (w/v) casein, 3.1 mM NaN₃, 10 mM ethyenediaminetetra-acetic acid (EDTA), and 0.05% (w/v) Tween 20 at pH 7.2-7.4. The sample is suspended in a boiling bath for 10 minutes and then centrifuged for 5 minutes at 10,000 g and the supernatant can be stored frozen at −70° C. until assayed for enterostatin.

The stored aliquots of serum can be later thawed at room temperature, thoroughly mixed and centrifuged for five minutes at 10,000 g. To extract enterostatin, the supernatant is mixed with 1:9 volume methanol. The mixture can be stored over ice for 30-60 minutes and then centrifuged at 11,000 g for 10 minutes at 4° C. The clear supernatant can be lyophilized and suspended in ELISA buffer for assay or in TBS (50 mM Tris.HCl, 150 nM NaCl, 3.1 mM NaN₃, pH 7.4) for chromatography. To inhibit proteolytic degradation of enterostatins during the assay, two protease inhibitors can be added to the serum samples before ELISA (final concentration, 1 mmol/L diprotein A and 0.1 mmol/L captopril).

Enzyme-Linked Immunosorbent Assay (ELISA)

Antibodies against APGPR can be generated utilizing standard techniques well know to those skill in the art. Antibodies can be polyclonal or monoclonal.

Preparation of Coating Antigen: 1 ml of a 5 mg bis (sulfosuccinimidyl) suberate (BS) (Pierce, Ill., USA) in PBS, pH 7.2 is slowly added dropwise to 2 ml of 10 g/l rabbit serum albumin (RSA) in PGS and stirred for 2 hours at room temperature. Excess BS is removed by gel filtration in PBS on a 2-20 cm Sephadex G-50 Column. The protein peak, as monitored by absorbance at 280 nm, is pooled. This is incubated overnight at 4° C. with two changes of dialysis buffer. The protein content is measured using a Lowry method (See e.g., Markwell et al., 1978, Anal. Biochem. 87:206-210) as 730 μg/ml This is diluted to 0.5 mg/ml and 3.1 mmol/l NaN₃ is added. Portions are stored at −20° C. till required.

Competitive ELISA: the wells of PVC microtitre plates are coated with 100 μl of 0.2 μg/ml RSA-BS-CCG-APGPR and 0.8 μg/ml RSA in 15 mmol/l Na₂CO₃, 35 mmol/l NaHCO₃, 3.1 mmol/l NaN₃ pH 9.6 by incubation overnight at 4° C. All further incubation are done at room temperature on an agitator. The plates are then washed three times and blocked with wash buffer (20 mmol/l Tris/HCl, 75 mmol/l NaCl, 3.1 mmol/l NaN_{3.} 0.05% (w/v) Tween 20 at pH 7.2-7.4). Then 100 μl of either unknown or standard synthetic APGPR peptide (purchased from American Peptide Company) solutions plus 50 μl 1:2000 mouse anti-APGPR monoclonal antibody in ELISA buffer (50 mM TRIS/HCL, 0.05% (w/v) casein, 3.1 mM NaN₃, 10 mM ethyenediaminetetra-acetic acid (EDTA), and 0.05% (w/v) Tween 20 at pH 7.2-7.4) are incubated in the wells for one hour. Between each incubation the plates are washed three times in wash buffer. Firstly 100 μl of 1:1000 goat anti-mouse IgG biotin conjugate in ELISA buffer is incubated in each well for 30 minutes, then 100 μl of 1:500 extravidin alkaline phosphatase solution in wash buffer is incubated in each well for 30 minutes. Finally 100 μl of 1 mg/ml p-nitrophenyl phosphate in substrate buffer (10% (w/v) diethanolamine/HCl, 0.49 mmol/l MgCl₂, 3.1 mmol/l NaN₃, pH 9.8) is incubated in each well until the maximum absorbance at 405 nm measured on an Anthos 2001 ELISA plate reader is 1.5 for the minimum standard peptide concentration. Reaction is terminated by adding 3 mmol/l NaOH (50 μl). The plate then read at 405 nm and a standard curve is constructed to calibrate the readings.

A standard inhibition curve under the specified conditions is obtained by plotting the concentration of competing synthetic antigen (APGPR) on the x axis, which is a log scale, against absorbance on they axis, which is a linear scale. The concentration of antigen (APGPR) in a sample from a subject can be interpolated from the standard antigen-inhibition curve.

Chromatography Analysis

To ascertain the size of serum APGPR immunoreactivity, gel filtration chromatography is performed using Sephadex G-25 (50×1.0 m; 9.3 ml; fractionation range for globular proteins, 1-5 kDa) column. Lyophilized methanol-extracted serum reconstituted in a minimal volume of distilled water is loaded on columns equilibrated with buffer A (10 mmol/l NH₄HCO₃). The column is eluted with 10 mmol/l NH₄HCO₃ at a rate of about 5 minute/1 ml fraction.

### 5.2. Example 2

### Oral Dosage Forms of Enterostatin

This example provides oral dosage forms comprising enterostatin or co-complexes comprising enterostatin.

Enterostatin is produced under Good Manufacture Procedures (cGMP) by American Peptide Company. The purity of enterostatin is greater than 95% by HPLC analysis.

Enterostatin co-complexes can be prepared as described in U.S. provisional application No. 60/750,208, filed Dec. 13, 2005, the contents of which are incorporated by reference in its entirety. For example, enterostatin co-complexes by combining a co-crystal guest and an enterostatin in a 1:1 molar ratio in a solvent. The solvents are allowed to evaporate and the resulting solid co-complexes is collected. The solvent can be methanol, the salt is enterostatin acetate and the guest is 1-hydroxy-2-naphthoic acid. The resulting solid is in the form of light brown flakes or broken glass.

Oral dosage forms of enterostatin may contain 2.5, 4, 5, 7.5, 10, 15, 20, 30, 40, 50, 60, or 70 mg enterostatin. They may comprise excipients or non-hygroscopic additives. Suitable excipients may be starches, sugars, and micro-crystalline cellulose etc. Suitable non-hygroscopic may be dibasic calcium phosphate anhydrous, calcium sulfate, powdered cellulose, dextrose and lactitol etc. Oral dosage forms of enterostatin may be in the form of tablets or capsules.

Exemplary capsules comprising enterostatin may contain a fill with 2.5% enterostatin (% weight), 42% Cremphor EL, 20% Labrasol, and 30% labrafil M2125CS, and a shell with 54% Gelatin, 18% Glycerin, 22% anidrisorb 35/70, and 6% water.

### 5.3. Example 3

### Treating Obesity with Enterostatin

This example provides treating subjects for obesity with oral dosage forms of enterostatin described in Example 2. The subjects are selected based on the amount of enterostatin in a sample from the subjects and a normal enterostatin value. In this example, the normal enterostatin value is established from a plurality of control subjects.

Selecting a Patient for Treating Obesity with Enterostatin

Determining Normal Enterostatin Value from Control Subjects

A physical examination, electrocardiogram, chemistry panel test, complete blood count and urinalysis are performed on men, aged 18-60 years, with a BMI from 20 kg/m² to 25 kg/m², according to the judgment of those of skill in the art. Based on these test and medical history, ten healthy men with normal BMI are chose to determine the normal enterostatin value. Subjects who are taking regular medications are excluded from the study.

These subjects are fasted over night and are allowed water but no caloric beverages. Next morning, a high fat meal is presented to these subjects. The high fat meal contains about 800 cal and contains 45% fat. About 100 to about 200 minutes, preferably 180 minutes after the meal is presented, 5 ml samples of blood are collected via an indwelling catheter from the subjects. Each blood sample is prepared and processed as described in Example 1. The amount of enterostatin from each blood sample is determined according to Example 1.

The normal enterostatin value for fasted subjects is calculated as means of the amount of enterostatin of samples taken from these subjects.

Selecting Subjects for Treating Obesity with Enterostatin

Ten obese (BMI from 30 kg/m² to 40 kg/m²), otherwise healthy men aged 18-60 years are screened for treatment with enterostatin. The health condition of these subjects are determined by physical examination and chemistry panel test etc. as described above for control subjects.

These obese subjects are fasted and fed, blood samples are taken as described above. The amount of enterostatin from each blood sample is determined using ELISA according to Example 1 and is compared to the normal enterostatin value determined above.

A subject is selected for treatment with enterostatin if the amount of enterostatin from his blood sample is less than half of the normal enterostatin value determined above. Capsules for oral administration comprising 20.0 mg enterostatin are prepared as described above. The enterostatin capsules are given to the selected subject at the start of three meals per day for four weeks.

Electrocardiogram, chemistry panel test, complete blood count and urinalysis are performed to monitor the safety of oral enterostatin administration. Food intake, energy intake, appetite, feeling of fullness, body weight, BMI, percentage of body fat and metabolic effects (such as sleeping metabolic rate, resting metabolic rate, fat oxidation and fat balance) etc. are monitored by practitioners of skill to determine the effectiveness and safety of oral enterostatin administration.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating or preventing a disorder or condition associated with enterostatin deficiency in a subject in need thereof, comprising administering to an enterostatin-deficient subject an amount of enterostatin effective for treating or preventing the disorder or condition.

2. The method of claim 1, wherein the disorder or condition is selected from overweight, obesity, metabolic disorders, hypertension, lipid related disorders, type II diabetes.

3.-26. (canceled)

27. A method of treating an obese or overweight human with enterostatin, comprising: a) determining the amount of enterostatin in a sample from the obese or overweight human, wherein the sample is obtained after the obese or overweight human is fed; b) selecting the obese or overweight human only if the amount of enterostatin in the sample of the obese or overweight human is less than 50% of a normal enterostatin value; and c) administering enterostatin to the selected obese or overweight human.

28. (canceled)

29. The method of claim 27, wherein the sample is selected from a blood sample, a plasma sample, a saliva sample, a serum sample, a sputum sample, a urine sample, a cell sample, a cellular extract sample and a tissue biopsy sample.

30. The method of claim 29, wherein the sample is a blood sample.

31.-32. (canceled)

33. The method of claim 27, wherein the amount of enterostatin in the sample from the obese or overweight human is determined by immunoassay.

34.-41. (canceled)

42. A method of reducing food intake in an enterostatin-deficient human in need thereof, comprising (a) determining the amount of enterostatin in a sample from a human; (b) selecting the human only if the amount of enterostatin in the sample from the human is less than 50% a normal enterostatin value; and (c) administering to the human an amount of enterostatin effective for reducing food intake.

43.-48. (canceled)

49. The method of claim 42, wherein the human has a Body Mass Index (“BMI”) greater than 30 kg/m².

50. The method of claim 42, wherein the human has a Body Mass Index (“BMI”) greater than 35 kg/m².

51.-54. (canceled)

55. The method of claim 42, wherein the sample is selected from a blood sample, a plasma sample, a saliva sample, a serum sample, a sputum sample, a urine sample, a cell sample, a cellular extract sample and a tissue biopsy sample.

56. The method of claim 55, wherein the sample is a blood sample.

57. (canceled)

58. The method of claim 42, wherein the amount of enterostatin in the sample from the human is determined spectrometry, chromatography, immunoassay or electrophoresis.

59-62. (canceled)

63. The method of claim 42, wherein the amount of enterostatin is determined when the human is fasted.

64. The method of claim 42, wherein the amount of enterostatin is determined when the human is fed.

65.-68. (canceled)

69. The method of claim 42, wherein said enterostatin is a peptide having an amino acid selected from the group consisting of APGPR (SEQ ID NO:1), VPDPR (SEQ ID NO:2) and VPGPR (SEQ ID NO:3).

70.-78. (canceled)

79. The method of claim 42, wherein enterostatin is administered in an amount of about 2 mg/day to about 40 mg/day.

80. The method of claim 42, wherein enterostatin is administered around a meal time.

81.-83. (canceled)

84. The method of claim 42, wherein the enterostatin is in an oral dosage form.

85. The method of claim 42, wherein the enterostatin is administered more than two times a day.
